(19) United States
(12) Patent Application Publication
(10) Pub. No.: US 2021/0289816 A1
(43) Pub. Date: Sep. 23, 2021

(54) ARTIFICIAL DIET FOR ORIUS STRIGICOLLIS, PREPARATION METHOD AND USE THEREOF

(71) Applicant: Zhejiang Academy of Agricultural Sciences, Zhejiang (CN)

(72) Inventors: Jun HUANG, Zhejiang (CN); Yaobin LV, Zhejiang (CN); Chendi YU, Zhejiang (CN); Cheng QIAN, Zhejiang (CN); Xiaowei LI, Zhejiang (CN); Zhijun ZHANG, Zhejiang (CN); Jinming ZHANG, Zhejiang (CN)

(21) Appl. No.: 16/893,715

(22) Filed: Jun. 5, 2020

(30) Foreign Application Priority Data
Mar. 20, 2020 (CN) 202010200512.8

(51) Int. Cl. A23K 20/142; A23K 50/90; A23K 20/174; A23K 20/22; A23K 20/24; A23K 20/163; A23K 40/00

(52) U.S. Cl. CPC A23K 20/142; A23K 50/90; A23K 20/174; A23K 40/00; A23K 20/24; A23K 20/163; A23K 20/22

(57) ABSTRACT

The present invention provides an artificial diet for Orius strigicollis (O. strigicollis), a preparation method and use thereof belonging to the technical field of insect rearing wherein the artificial diet includes the following raw materials: compound amino acids, water, potassium sorbate, vitamin complex, folic acid, biotin, glucose, sodium chloride, yeast powder, sodium alginate, calcium lactate, and greenish yellow food coloring. The artificial diet of the present invention is beneficial for O. strigicollis to forage, promotes growth and development thereof, improves fecundity thereof, and increases the survival rate of O. strigicollis nymphs.

### CROSS REFERENCE TO RELATED APPLICATIONS

The present application claims the benefit of priority under 35 USC 119 to Chinese patent application 202010200512.8, filed Mar. 20, 2020, the contents of which are incorporated herein by reference.

### TECHNICAL FIELD

The present invention relates to the technical field of insect rearing, and in particular to an artificial diet for Orius strigicollis, a preparation method and use thereof.

### BACKGROUND

Orius strigicollis (Hemiptera: Anthocoridae) is a dominant insect species in South China. Because of strong predation capacity, nymphs and adults of this species have significant control effects on both the eggs and the newly hatched larvae of thrips, aphids, spider mites, whiteflies, leafhoppers, and lepidopterous pests. Accordingly, this insect is a promising and beneficial predator. So far, diets for O. strigicollis include animal-derived, plant-derived, and artificial diets. In particular, the majority of artificial diets for O. strigicollis are solid or liquid. O. strigicollis is a piercing sucking insect. Therefore, solid diets are inconvenient for O. strigicollis to forage which is contrary to the growth and development of O. strigicollis. On the other hand, liquid diets often cause low instar nymphs to die from sticking. Therefore, it is now necessary to develop an artificial diet which not only promotes the growth and development of O. strigicollis, but also reduces the mortality of low instar nymphs.

### SUMMARY

An objective of the present invention is to provide an artificial diet for Orius strigicollis (O. strigicollis), a preparation method and use thereof. The artificial diet of the present invention favors the growth and development of O. strigicollis and reduces the mortality of low instar nymphs.

To achieve the above purpose, the present invention provides the following technical solutions.

An artificial diet for Orius strigicollis according to this invention includes the following raw materials: 6 to 10 parts by weight of compound amino acids, 180 to 220 parts by weight of water, 0.001 to 0.005 part by weight of potassium sorbate, 0.05 to 0.15 part by weight of vitamin complex, 0.05 to 0.15 part by weight of folic acid, 0.05 to 0.15 part by weight of biotin, 5 to 15 parts by weight of glucose, 0.5 to 1 part by weight of sodium chloride, 0.5 to 1 part by weight of yeast powder, 1 to 1.5 parts by weight of sodium alginate, 2 to 3 parts by weight of calcium lactate, and 0.5 to 1.0 part by weight of greenish yellow food coloring; wherein:

the compound amino acids include the following components: 4 to 5 percent by weight of alanine, 35 to 40 percent by weight of arginine, 6 to 7 percent by weight of glutamine, 1.5 to 2 percent by weight of glycine, 12 to 13 percent by weight of histidine, 2 to 3 percent by weight of isoleucine, 1.5 to 2 percent by weight of leucine, 6 to 6.5 percent by weight of lysine, 3 to 3.5 percent by weight of phenylalanine, 3 to 4 percent by weight of proline, 4 to 5 percent by weight of serine, 3 to 4 percent by weight of threonine, 6 to 6.5 percent by weight of tryptophan, 2 to 3 percent by weight of valine, and 2.8 to 3.5 percent by weight of asparagine.

The artificial diet of this invention is prepared as a spherical semisolid pellet feed; and it is sieved with a 100-mesh sieve.

In an embodiment of the invention, the compound amino acids include the following components: 4.5 percent by weight of alanine, 37.3 percent by weight of arginine, 6.8 percent by weight of glutamine, 1.8 percent by weight of glycine, 12.6 percent by weight of histidine, 2.5 percent by weight of isoleucine, 1.9 percent by weight of leucine, 6.2 percent by weight of lysine, 3.2 percent by weight of phenylalanine, 3.8 percent by weight of proline, 4.4 percent by weight of serine, 3.4 percent by weight of threonine, 6.2 percent by weight of tryptophan, 2.4 percent by weight of valine, and 3 percent by weight of asparagine.

A preparation method of the above artificial diet may include the following steps:

1) mixing the compound amino acids, potassium sorbate, vitamin complex, folic acid, biotin, glucose, sodium chloride, yeast powder, and sodium alginate with part of the water to obtain a nutrient solution;

2) mixing calcium lactate and greenish yellow food coloring with the remaining water to obtain a calcium lactate-food coloring solution; and

3) adding the nutrient solution of Step 1) dropwise to the calcium lactate-food coloring solution of Step 2) to form semisolid pellets, passing the pellets through a 100-mesh sieve, collecting the semisolid pellets from the sieve and immersing them in water, and dehydrating the immersed semisolid pellets to obtain the artificial diet; wherein:

a mass ratio of the part of the water used in Step 1) to the remaining water used in Step 2) is (0.8-1.2):(0.8-1.2); and,

Step 1) and Step 2) of the method can be performed in any sequence or at the same time.

In an embodiment, a temperature of the mixing of Step 1) is 22 to 28° C.

In an embodiment, the duration between the end of the dropwise addition and the pre-sieving of Step 3) is no longer than 15 s.

In an embodiment, the time of the immersion of Step 3) is 1 to 3 min.

The present invention further provides use of the above artificial diet in the promotion of the growth and development of Orius strigicollis.

The present invention further provides use of the above artificial diet in the improvement of the survival rate of Orius strigicollis nymphs.

The present invention further provides use of the above artificial diet in the improvement of fecundity of Orius strigicollis.

In an embodiment, the use includes the following steps: feeding the artificial diet to at least first instar Orius strigicollis (O. strigicollis) for rearing wherein: a quantity ratio of the artificial diet fed each time to the O. strigicollis is in a range of 3:1 to 5:1 when the O. strigicollis is at the first to the third instar larval stage, and a quantity ratio of the artificial diet fed each time to the O. strigicollis is in a range of 2:1 to 3:1 when the O. strigicollis is at the fourth instar larval stage and above; and, changing the artificial diet every three days.

Among the benefits of the present invention are the following: The present invention provides an artificial diet for O. strigicollis, including the following raw materials: compound amino acids, water, potassium sorbate, vitamin complex, folic acid, biotin, glucose, sodium chloride, yeast powder, sodium alginate, calcium lactate, and greenish yellow food coloring. The compound amino acids include the following components: alanine, arginine, glutamine, glycine, histidine, isoleucine, leucine, lysine, phenylalanine, proline, serine, threonine, tryptophan, valine, and asparagine. The artificial diet is a spherical semisolid pellet feed. The artificial diet is sieved with a 100-mesh sieve. In the present invention, the compound amino acids supplement all amino acid O. strigicollis needs and provide O. strigicollis with protein raw materials. A nutrient solution composed of the compound amino acids, the vitamin complex, the folic acid, the biotin, the glucose, the sodium chloride, the yeast powder, the sodium alginate, and the water provides adequate nutrition for the growth and development of O. strigicollis. In the present invention, the artificial diet is a spherical semisolid pellet feed that has been sieved with a 100-mesh sieve. Compared with conventional solid artificial diets, the artificial diet of this invention is more beneficial for O. strigicollis to forage, promotes growth and development thereof, improves fecundity thereof, and increases the survival rate of O. strigicollis nymphs.

### DETAILED DESCRIPTION

The present invention provides an artificial diet for Orius strigicollis (O. strigicollis), including the following raw materials: 6 to 10 parts by weight of compound amino acids, 180 to 220 parts by weight of water, 0.001 to 0.005 part by weight of potassium sorbate, 0.05 to 0.15 part by weight of vitamin complex, 0.05 to 0.15 part by weight of folic acid, 0.05 to 0.15 part by weight of biotin, 5 to 15 parts by weight of glucose, 0.5 to 1 part by weight of sodium chloride, 0.5 to 1 part by weight of yeast powder, 1 to 1.5 parts by weight of sodium alginate, 2 to 3 parts by weight of calcium lactate, and 0.5 to 1.0 part by weight of greenish yellow food coloring. In an embodiment of the invention, the artificial diet includes the following raw materials: 8.44 parts by weight of compound amino acids, 200 parts by weight of water, 0.003 part by weight of potassium sorbate, 0.1 part by weight of vitamin complex, 0.1 part by weight of folic acid, 0.1 part by weight of biotin, 10 parts by weight of glucose, 0.9 part by weight of sodium chloride, 0.6 part by weight of yeast powder, 1.12 parts by weight of sodium alginate, 2.55 parts by weight of calcium lactate, and 0.6 part by weight of greenish yellow food coloring.

In the present invention, the compound amino acids include the following components: 4 to 5 percent by weight of alanine, 35 to 40 percent by weight of arginine, 6 to 7 percent by weight of glutamine, 1.5 to 2 percent by weight of glycine, 12 to 13 percent by weight of histidine, 2 to 3 percent by weight of isoleucine, 1.5 to 2 percent by weight of leucine, 6 to 6.5 percent by weight of lysine, 3 to 3.5 percent by weight of phenylalanine, 3 to 4 percent by weight of proline, 4 to 5 percent by weight of serine, 3 to 4 percent by weight of threonine, 6 to 6.5 percent by weight of tryptophan, 2 to 3 percent by weight of valine, and 2.8 to 3.5 percent by weight of asparagine. In an embodiment of the invention, the compound amino acids include the following components: 4.5 percent by weight of alanine, 37.3 percent by weight of arginine, 6.8 percent by weight of glutamine, 1.8 percent by weight of glycine, 12.6 percent by weight of histidine, 2.5 percent by weight of isoleucine, 1.9 percent by weight of leucine, 6.2 percent by weight of lysine, 3.2 percent by weight of phenylalanine, 3.8 percent by weight of proline, 4.4 percent by weight of serine, 3.4 percent by weight of threonine, 6.2 percent by weight of tryptophan, 2.4 percent by weight of valine, and 3 percent by weight of asparagine. In the present invention, the compound amino acids supplement all amino acid O. strigicollis needs and provide O. strigicollis with protein raw materials.

In the present invention, the potassium sorbate functions as a preservative. The potassium sorbate may be in granular form, as C₆H₇O₂K, ranging from 98.0 to 101.0 percent by weight on a dry basis.

In an embodiment of the present invention, 0.735 g of the vitamin complex consists of 501 μg of vitamin A, 80 μg of vitamin C, 5 μg of vitamin D, 12 mg of vitamin E, 25 μg of vitamin K, 12.6 mg of vitamin B6, 126m of vitamin B12, 50 mg of pantothenic acid, 354 mg of calcium, 1.578 mg of phosphorus, 15 mg of zinc, 1.5 mg of manganese, 80 μg of selenium, and 100 μg of chromium, thereby providing O. strigicollis with a plurality of vitamins, minerals, and trace elements.

In the present invention, the folic acid is preferably analytically pure folic acid.

In the present invention, the biotin is preferably analytically pure biotin. The role of the biotin is to provide O. strigicollis with vitamin H. The vitamin H is necessary for vitamin C synthesis and essential to normal fat and protein metabolism.

In the present invention, the glucose is preferably analytically pure glucose. The role of the glucose is to provide an energy substance.

In the present invention, the sodium chloride is preferably analytically pure sodium chloride. The role of the sodium chloride is to provide sodium and chlorine so as to satisfy nutritional requirements of O. strigicollis.

In the present invention, the yeast powder functions as a nutritional supplement.

In the present invention, the artificial diet is a spherical semisolid pellet feed that has been sieved with a 100-mesh sieve. The artificial diet has a water content of preferably 45% to 65%, and more preferably 55%.

The present invention further provides a preparation method for preparing the artificial diet including the following steps:

1) mixing the compound amino acids, potassium sorbate, vitamin complex, folic acid, biotin, glucose, sodium chloride, yeast powder, and sodium alginate with part of the water to obtain a nutrient solution;

2) mixing calcium lactate and greenish yellow food coloring with the remaining water to obtain a calcium lactate-food coloring solution; and

3) adding the nutrient solution of Step 1) dropwise to the calcium lactate-food coloring solution of Step 2) to form semisolid pellets, passing the pellets through a 100-mesh sieve, collecting the semisolid pellets from the sieve and immersing them in water, and dehydrating the immersed semisolid pellets to obtain the artificial diet; wherein:

a mass ratio of the part of the water used in Step 1) to the remaining water used in Step 2) is (0.8-1.2):(0.8-1.2), and preferably 1:1; and

Step 1) and Step 2) of the method can be performed in any sequence or at the same time.

In the present invention, the compound amino acids, the potassium sorbate, the vitamin complex, the folic acid, the biotin, the glucose, the sodium chloride, the yeast powder, and the sodium alginate are first mixed with the part of the water to obtain the nutrient solution.

In implementing an embodiment of the present invention, an aqueous compound amino acids solution is obtained by a first mixing step comprising mixing the compound amino acids with part of the water. Next, in a second mixing step, the potassium sorbate, the vitamin complex, the folic acid, the biotin, the glucose, the sodium chloride, and the yeast powder are mixed into the aqueous compound amino acids solution to obtain a primary nutrient solution. Then, in a third mixing step, four aliquots of the sodium alginate are charged into the primary nutrient solution to obtain the completed nutrient solution.

In the present invention, the first mixing step, the second mixing step, and the third mixing step are preferably conducted at a temperature of 22 to 28° C., and more preferably at 25° C., respectively. The time and method of the first mixing step, the second mixing step, and the third mixing step are not critical in the present invention as long as the mixing of each step is thorough and homogeneous. In implementing the present invention, the third mixing step is preferably conducted by heating the mixture in a water bath at 25° C., to minimize any problems of insolubility due to clotting. The sodium alginate is stirred for 30 min or until dissolved thoroughly, and the completed nutrient solution is thereby obtained.

In the present invention, the calcium lactate and the greenish yellow food coloring are mixed with the remaining water to obtain the calcium lactate-food coloring solution.

In implementing the present invention, an aqueous solution of calcium lactate is obtained by a fourth mixing step of mixing the calcium lactate with the remaining water. Next, the calcium lactate-food coloring solution is obtained by a fifth mixing step of mixing the aqueous solution of calcium lactate with the greenish yellow food coloring. The fourth mixing step is preferably conducted at a temperature of 20 to 30° C., and more preferably at 25° C. The fourth mixing step is preferably carried out by stirring and mixing for preferably about 3 min. The fifth mixing step is preferably conducted at a temperature of 20 to 30° C., and more preferably at 25° C. The fifth mixing step is preferably carried out by stirring and mixing for preferably about 15 s.

After the completed nutrient solution and the calcium lactate-food coloring solution are obtained, the nutrient solution is added dropwise (e.g., at a rate of 180 gtt/min) to the calcium lactate-food coloring solution. The mixture is sieved using a 100-mesh sieve. The semisolid pellets are collected from the sieve and immersed in water. The semisolid pellets are then dehydrated to obtain the artificial diet. The time between the end of the dropwise addition step and the sieving step is preferably no greater than 15 s, and more preferably no greater than 5 s. The time of the water immersion is preferably 1 to 3 min, and more preferably 2 min. In implementing the present invention, a 20 mL syringe without needle containing 20 mL of the above solution is aspirated to drip into the calcium lactate-food coloring solution in a teardrop manner. Semisolid pellets will be produced immediately after the teardrop nutrient solution is exposed to the calcium lactate solution according to the following chemical reaction equation: (C₆H₇O₆Na)n+C₆H₁₀CaO₆.5H₂O+H⁺=C₁₈H₂₄CaO₁₉+C₃H₅O₃Na+H₂). Preferably, semisolid pellets are sieved with a gauze, and the gauze with semisolid pellets is immersed in distilled water. The dehydration of the immersed semisolid pellets may be carried out by placing the gauze on a piece of dry tissue for 5 s to absorb the water on the surfaces of the semisolid pellets so as to obtain the artificial diet for O. strigicollis.

In the present invention, the artificial diet for O. strigicollis is stored in a dry and pollution-free Petri dish, sealed with a sealing film, and then stored at 4 to 8° C., with a shelf life of 5 to 10 days.

The present invention further provides for using the artificial diet prepared as described above in promoting the growth and development of O. strigicollis.

The present invention further provides for using the artificial diet prepared as described above in improving the survival rate of O. strigicollis nymphs.

The present invention further provides for using the artificial diet prepared as described above in improving fecundity of O. strigicollis. The improvement of fecundity of O. strigicollis preferably includes prolongation of an oviposition period and improvement of an oviposition amount.

In the present invention, the use of the artificial diet preferably includes the following steps: feeding the artificial diet to at least first instar O. strigicollis for rearing wherein a quantity ratio of the artificial diet fed each time to the O. strigicollis is in a range of 3:1 to 5:1 and preferably is 4:1 when the O. strigicollis is at the first to the third instar larval stage, and a quantity ratio of the artificial diet fed each time to the O. strigicollis is in a range of 2:1 to 3:1 and preferably is 2.5:1 when the O. strigicollis is at the fourth instar larval stage and above; and, changing the artificial diet every three days.

In the present invention, the rearing temperature is preferably maintained at 27° C., humidity is preferably maintained at 70%, and light conditions are preferably maintained on a 14 h light/10 h dark cycle. A container for the rearing is preferably a glass rearing jar. In implementing the present invention, rearing jar and rearing environment conditions may be adapted according to technical solutions described in INDOOR EXPANDING PROPAGATION METHOD FOR ORIUS STRIGICOLLIS (Chinese Patent Application No. CN201910857206.9) by HUANG Jun et al.

The present invention will be more clearly and completely described below in conjunction with several embodiments of the present invention. It should be understood, however, that the following embodiments are merely illustrative of some rather than all of the embodiments of the present invention. All other embodiments obtained by a person of ordinary skill in the art based on the embodiments of the present invention herein without creative efforts shall be considered to be within the scope of protection of the present invention.

### EXAMPLE 1

### Preparation of an Artificial Diet for Orius strigicollis

Weighing and dissolving of 15 amino acids: Various amino acids were weighed successively by an electronic scale (as shown in Table 1); a total of 8.44 g of amino acids weighed were charged into a 250 mL beaker, mixed with 100 mL of distilled water at 25° C., stirred with a glass rod for 5 min until dissolved thoroughly to obtain a clear solution. This portion could provide Orius strigicollis with the most important components of proteins.

TABLE 1
Weights (in gram) and weight percent of 15 amino acids

| No. | Name of amino acid | Weight percent | Weight (g) |
|---|---|---|---|
| 1 | Alanine | 4.5% | 0.38 |
| 2 | Arginine | 37.3% | 3.15 |
| 3 | Glutamine | 6.8% | 0.57 |
| 4 | Glycine | 1.8% | 0.15 |
| 5 | Histidine | 12.6% | 1.06 |
| 6 | Isoleucine | 2.5% | 0.21 |
| 7 | Leucine | 1.9% | 0.16 |
| 8 | Lysine | 6.2% | 0.52 |
| 9 | Phenylalanine | 3.2% | 0.29 |
| 10 | Proline | 3.8% | 0.32 |
| 11 | Serine | 4.4% | 0.37 |
| 12 | Threonine | 3.4% | 0.29 |
| 13 | Tryptophan | 6.2% | 0.52 |
| 14 | Valine | 2.4% | 0.2 |
| 15 | Asparagine | 3% | 0.25 |

The following raw materials were charged into the foregoing solution successively:

potassium sorbate 0.003 g (granule, as C6H₇O2K, 98.0 to 101.0 w% on a dry basis): this component functions as a preservative;

vitamin complex 0.1 g (which includes vitamins A, C, D, E, K, B1, B2, B6, and B12, pantothenic acid, calcium, iron, magnesium, zinc, manganese, selenium, and chromium): this component supplements vitamins, minerals and trace elements;

analytically pure folic acid 0.1 g;

analytically pure biotin 0.1 g: this component provides O. strigicollis with vitamins;

analytically pure glucose 10 g: this component provides an energy substance;

analytically pure sodium chloride 0.9 g: this component supplements sodium and chlorine so as to satisfy nutritional requirements of O. strigicollis; and

yeast powder 0.6 g: this component functions as a nutritional supplement.

The above raw materials were stirred at 25° C. until dissolved thoroughly.

Four aliquots of pure sodium alginate (1.12 g in total) are charged into the foregoing solution, while heating in a water bath at 25° C., in case of insolubility due to clotting. The solution was stirred for 30 min until the sodium alginate was dissolved thoroughly, and a nutrient solution was obtained for use.

One hundred milliliters of distilled water was charged into a second 250 mL beaker, then mixed with 2.55 g of analytically pure calcium lactate, and stirred with a glass rod for 3 min at room temperature until dissolved thoroughly; finally, 0.1 g of greenish yellow food coloring was added and stirred well for 15 s to make the solution appear greenish yellow.

Using a 20 mL syringe without needle, 20 mL of the above nutrient solution was aspirated; while pushing the syringe slowly, the nutrient solution pushed out was dripped into the calcium lactate solution in a teardrop manner; semisolid pellets were produced immediately after the teardrop nutrient solution was exposed to the calcium lactate solution; within 5 s, the semisolid pellets were filtered using a 100-mesh gauze; subsequently, the gauze with semisolid pellets was immersed in distilled water for 2 min; then, the gauze was placed on a piece of dry tissue for 5 s to absorb the water on the surface of semisolid pellets, so as to obtain the artificial diet for O. strigicollis.

### EXAMPLE 2

### Storage of the Artificial Diet of Example 1

After preparation, the spherical semisolid artificial diet was stored in a dry and pollution-free Petri dish, sealed with a sealing film, and stored in a refrigerator at 4° C., with a shelf life of 10 days.

### EXAMPLE 3

### Usage and Benefits of the Artificial Diet of Example 1

### 1. Usage

An Orius strigicollis (O. strigicollis) rearing workshop had a temperature of 27° C., a humidity of 70%, and a 14 h light/10 h dark cycle. The artificial diet for O. strigicollis was fed in glass rearing jars. For the rearing jars and rearing environment conditions used in this Example, refer to the description in INDOOR EXPANDING PROPAGATION METHOD FOR ORIUS STRIGICOLLIS (Chinese Patent Application No. CN201910857206.9) by HUANG Jun et al.

1) A piece of 10 by 10 cm weighing paper was placed flatwise in a rearing jar; after a Petri dish with artificial diet was uncovered, the artificial diet was tweezered gently or tapped onto the weighing paper.

2) Once 2^{nd} instar O. strigicollis appeared in the rearing jar, they were fed the artificial diet of Example 1. A quantity ratio of the artificial diet to the O. strigicollis was 3:1. Once at least 4^{th} instar O. strigicollis appeared in the rearing jar, the quantity ratio of the artificial diet to the O. strigicollis was adjusted to 2:1, and they were reared until the adult stage.

3) The artificial diet was changed every three days, i.e., the whole piece of weighing paper and the residual artificial diet thereon were changed and replaced with fresh weighing paper and fresh artificial diet.

### 2. Benefits of the Present Invention

O. strigicollis nymphs (nymphs were similar in size and color and were fasted for 24 h; 10 nymphs each group) and adults (two consecutive generations) were reared with the spherical semisolid artificial diet of the present invention. Measurements were made of growth and development indices at all growth stages of O. strigicollis. In Control Group 1, O. strigicollis were fed with thrips, the optimal preys. In Control Group 2, O. strigicollis (which were reared with pure animal protein and could complete generations) were fed with rice moth eggs. In Control Group 3, 0. strigicollis were fed with a spherical semisolid artificial diet but without the 15 amino acids.

In comparing the artificial diet group according to the present invention with Control Group 1, differences in developmental duration and survival rate are not significant among nymphs from all instars (Tables 2 and 3), which indicates that the artificial diet fully satisfies nutritional requirements during the development of O. strigicollis nymphs from all instars, and also that the rearing effect is consistent with that of feeding using optimal preys. In comparing the artificial diet group according to the present invention with Control Group 2, the developmental duration of low instar nymphs (1^{st} and 2^{nd} instars) reared using the artificial diet is significantly lower (i.e., better) than that of Control Group 2, and there are no significant differences in developmental duration between high instar nymphs reared in both groups (Table 2). Low instar nymphs (1^{st}, 2^{nd} and 3^{rd} instars) reared in the artificial diet group have a higher survival rate than Control Group 2, and there are no significant differences in survival rate between high instar nymphs reared in both groups (Table 3). Data were not analyzed because O. strigicollis fed with the spherical semisolid artificial diet without the 15 amino acids (Control Group 3) failed to develop normally. It can be seen from the survival rate listed in Table 3 and the experiment process that O. strigicollis have a high survival rate with the artificial diet of this invention.

TABLE 2
The developmental duration of two consecutive generations of
O. strigicollis nymphs after foraging of the artificial diet

| | Developmental duration of nymphs from all instars (d) | | | | |
|---|---|---|---|---|---|
| Treatment | 1^{st} instar | 2^{nd} instar | 3^{rd} instar | 4^{th} instar | 5^{th} instar |
| 1^{st} generation | 2.1 ± 0.1 b | 2.3 ± 0.2 b | 2.6 ± 0.2 a | 1.9 ± 0.1 a | 4.3 ± 0.1 a |
| 2^{nd} generation | 1.9 ± 0.1 b | 2.3 ± 0.2 b | 2.5 ± 0.1 a | 1.3 ± 0.1 a | 4.5 ± 0.2 a |
| Control 1 | 2.1 ± 0.1 b | 2.4 ± 0.1 b | 2.4 ± 0.1 a | 1.4 ± 0.2 a | 3.9 ± 0.3 a |
| Control 2 | 4.8 ± 0.6 a | 4.2 ± 0.3 a | 2.5 ± 0.2 a | 1.9 ± 0.2 a | 3.8 ± 0.3 a |
| Control 3 | — | — | — | — | — |

NOTE:
The number of test samples is 30 nymphs each group. The data listed in the table represent mean ± standard error. Different lowercase letters following the data in the same column represent a significant difference at a 0.05 level; otherwise, the difference is insignificant. Dashes (“—”) annotated in the table indicate that more than 90% of test subjects fail to develop normally and statistical analysis is not performed. (Hereinafter inclusive)

TABLE 3
The survival rate of two consecutive generations of O. strigicollis
nymphs after foraging of the artificial diet

| | Survival rate of nymphs from all instars (%) | | | | |
|---|---|---|---|---|---|
| Treatment | 1^{st} instar | 2^{nd} instar | 3^{rd} instar | 4^{th} instar | 5^{th} instar |
| 1^{st} generation | 80.00 ± 6.24 a | 93.33 ± 4.08 a | 70.00 ± 6.24 a | 66.67 ± 14.61 a | 80.00 ± 3.33 a |
| 2^{nd} generation | 86.67 ± 3.33 a | 90.00 ± 4.08 a | 73.33 ± 6.67 a | 73.33 ± 4.08 a | 83.33 ± 5.27 a |
| Control 1 | 86.67 ± 6.24 a | 93.33 ± 4.08 a | 80.00 ± 3.33 a | 73.33 ± 4.08 a | 80.00 ± 6.24 a |
| Control 2 | 60.00 ± 8.50 b | 66.67 ± 7.45 b | 50.00 ± 5.27 b | 66.67 ± 5.27 a | 73.33 ± 8.50 a |
| Control 3 | — | — | — | — | — |

From Table 4, first-generation adults of the artificial diet group experience a shorter oviposition period than those of Control Group 1, but the difference is insignificant compared with Control Group 2. The difference in oviposition amount is insignificant between artificial diet group and Control Group 1, but higher than Control Group 2. There is no significant difference in life-span of female adults among three groups; however, first-generation male adults of the artificial diet group live longer than those of Control Group 1, while the life-span of second-generation male adults increases significantly, without significant difference between artificial diet group and Control Group 1, but higher than Control Group 2.

TABLE 4
The fecundity of two consecutive generations of O. strigicollis
adults after foraging of the artificial diet

| | Oviposition | Oviposition | Life-span (day) | |
|---|---|---|---|---|
| Treatment | period (day) | amount (egg) | Female adult | Male adult |
| 1^{st} generation | 9.4 ± 2.2 b | 65.5 ± 10.6 a | 18.2 ± 1.2 a | 9.5 ± 0.9 b |
| 2^{nd} generation | 11.6 ± 0.8 b | 58.2 ± 4.2 a | 18.1 ± 1.1 a | 13.2 ± 1.2 a |
| Control 1 | 16.4 ± 3.2 a | 69.1 ± 3.3 a | 20.1 ± 1.4 a | 13.1 ± 0.9 a |
| Control 2 | 9.5 ± 0.7 b | 50.2 ± 1.2 b | 18.1 ± 0.5 a | 9.9 ± 0.6 b |
| Control 3 | — | — | — | — |

NOTE:
Each pair of male and female adults is a replicate. Fifteen replicates are set up. The data listed in the table represent mean ± standard error. Different lowercase letters following the data in the same column represent a significant difference at a 0.05 level; otherwise, the difference is insignificant.

The above descriptions are merely illustrative of some preferred implementations of the present invention. It should be understood that a person of ordinary skill in the art may make several improvements and modifications without departing from the principle of the present invention, and all such improvements and modifications shall also be deemed as falling within the scope of protection of the present invention.

What is claimed is:

1. An artificial diet for Orius strigicollis, prepared as a spherical semisolid pellet feed sieved with a 100-mesh sieve, comprising the following raw materials: 6 to 10 parts by weight of compound amino acids, 180 to 220 parts by weight of water, 0.001 to 0.005 part by weight of potassium sorbate, 0.05 to 0.15 part by weight of vitamin complex, 0.05 to 0.15 part by weight of folic acid, 0.05 to 0.15 part by weight of biotin, 5 to 15 parts by weight of glucose, 0.5 to 1 part by weight of sodium chloride, 0.5 to 1 part by weight of yeast powder, 1 to 1.5 parts by weight of sodium alginate, 2 to 3 parts by weight of calcium lactate, and 0.5 to 1.0 part by weight of greenish yellow food coloring; wherein
the compound amino acids comprise the following components: 4 to 5 percent by weight of alanine, 35 to 40 percent by weight of arginine, 6 to 7 percent by weight of glutamine, 1.5 to 2 percent by weight of glycine, 12 to 13 percent by weight of histidine, 2 to 3 percent by weight of isoleucine, 1.5 to 2 percent by weight of leucine, 6 to 6.5 percent by weight of lysine, 3 to 3.5 percent by weight of phenylalanine, 3 to 4 percent by weight of proline, 4 to 5 percent by weight of serine, 3 to 4 percent by weight of threonine, 6 to 6.5 percent by weight of tryptophan, 2 to 3 percent by weight of valine, and 2.8 to 3.5 percent by weight of asparagine.

2. The artificial diet according to claim 1, wherein the compound amino acids comprise the following components: 4.5 percent by weight of alanine, 37.3 percent by weight of arginine, 6.8 percent by weight of glutamine, 1.8 percent by weight of glycine, 12.6 percent by weight of histidine, 2.5 percent by weight of isoleucine, 1.9 percent by weight of leucine, 6.2 percent by weight of lysine, 3.2 percent by weight of phenylalanine, 3.8 percent by weight of proline, 4.4 percent by weight of serine, 3.4 percent by weight of threonine, 6.2 percent by weight of tryptophan, 2.4 percent by weight of valine, and 3 percent by weight of asparagine.

3. A preparation method for preparing the artificial diet according to claim 1, comprising the following steps:
1) mixing the compound amino acids, potassium sorbate, vitamin complex, folic acid, biotin, glucose, sodium chloride, yeast powder, and sodium alginate with part of the water to obtain a nutrient solution;
2) mixing calcium lactate and greenish yellow food coloring with the remaining water to obtain a calcium lactate-food coloring solution; and
3) adding the nutrient solution of Step 1) dropwise to the calcium lactate-food coloring solution of Step 2) to form semisolid pellets, passing the pellets through a 100-mesh sieve, collecting the semisolid pellets from the sieve and immersing them in water, and dehydrating the semisolid pellets to obtain the artificial diet; wherein:
a mass ratio of the part of the water used in Step 1) to the remaining water used in Step 2) is (0.8-1.2):(0.8-1.2); and
Step 1) and Step 2) of the method are performed in any sequence or at the same time.

4. A preparation method for preparing the artificial diet according to claim 2, comprising the following steps:
1) mixing the compound amino acids, potassium sorbate, vitamin complex, folic acid, biotin, glucose, sodium chloride, yeast powder, and sodium alginate with part of the water to obtain a nutrient solution;
2) mixing calcium lactate and greenish yellow food coloring with the remaining water to obtain a calcium lactate-food coloring solution; and
3) adding the nutrient solution of Step 1) dropwise to the calcium lactate-food coloring solution of Step 2) to form semisolid pellets, passing the pellets through a 100-mesh sieve, collecting the semisolid pellets from the sieve and immersing them in water, and dehydrating the semisolid pellets to obtain the artificial diet; wherein:
a mass ratio of the part of the water used in Step 1) to the remaining water used in Step 2) is (0.8-1.2):(0.8-1.2); and
Step 1) and Step 2) of the method are performed in any sequence or at the same time.

5. The preparation method according to claim 3, wherein the mixing in Step 1) is carried out at a temperature of 22 to 28° C.

6. The preparation method according to claim 4, wherein the mixing in Step 1) is carried out at a temperature of 22 to 28° C.

7. The preparation method according to claim 3, wherein the time between the end of the dropwise addition step and the sieving in Step 3) is no greater than 15 s.

8. The preparation method according to claim 4, wherein the time between the end of the dropwise addition step and the sieving in Step 3) is no greater than 15 s.

9. The preparation method according to claim 3, wherein the time of the pellet immersion in Step 3) is 1 to 3 min.

10. The preparation method according to claim 4, wherein the time of the pellet immersion in Step 3) is 1 to 3 min.

11. A method for promoting the growth and development of Orius strigicollis comprising a step of feeding them an artificial diet according to claim 1.

12. A method for promoting the growth and development of Orius strigicollis comprising a step of feeding them an artificial diet according to claim 2.

13. A method for improving the survival rate of Orius strigicollis nymphs comprising a step of feeding them an artificial diet according to claim 1.

14. A method for improving the survival rate of Orius strigicollis nymphs comprising a step of feeding them an artificial diet according to claim 2.

15. A method for improving the fecundity of Orius strigicollis comprising a step of feeding them an artificial diet according to claim 1.

16. A method for improving the fecundity of Orius strigicollis comprising a step of feeding them an artificial diet according to claim 2.

17. The method according to claim 11, wherein the method comprises the following steps:
feeding the artificial diet to at least first instar Orius strigicollis (O. strigicollis) for rearing wherein a quantity ratio of the artificial diet fed each time to the O. strigicollis is in the range of 3:1 to 5:1 when the O. strigicollis is at the first to the third instar larval stage, and a quantity ratio of the artificial diet fed each time to the O. strigicollis is in the range of 2:1 to 3:1 when the O. strigicollis is at the fourth instar larval stage and above; and, changing the artificial diet every three days.

18. The method according to claim 12, wherein the method comprises the following steps:
feeding the artificial diet to at least first instar Orius strigicollis (O. strigicollis) for rearing wherein a quantity ratio of the artificial diet fed each time to the O. strigicollis is in the range of 3:1 to 5:1 when the O. strigicollis is at the first to the third instar larval stage, and a quantity ratio of the artificial diet fed each time to the O. strigicollis is in the range of 2:1 to 3:1 when the O. strigicollis is at the fourth instar larval stage and above; and, changing the artificial diet every three days.

19. The method according to claim 13, wherein the method comprises the following steps:
feeding the artificial diet to at least first instar Orius strigicollis (O. strigicollis) for rearing wherein a quantity ratio of the artificial diet fed each time to the O. strigicollis is in the range of 3:1 to 5:1 when the O. strigicollis is at the first to the third instar larval stage, and a quantity ratio of the artificial diet fed each time to the O. strigicollis is in the rage of 2:1 to 3:1 when the O. strigicollis is at the fourth instar larval stage and above; and, changing the artificial diet every three days.

20. The method according to claim 15, wherein the method comprises the following steps:
feeding the artificial diet to at least first instar Orius strigicollis (O. strigicollis) for rearing wherein a quantity ratio of the artificial diet fed each time to the O. strigicollis is in the range of 3:1 to 5:1 when the O. strigicollis is at the first to the third instar larval stage and a quantity ratio of the artificial diet fed each time to the O. strigicollis is in the range of 2:1 to 3:1 when the O. strigicollis is at the fourth instar larval stage and above; and, changing the artificial diet every three days.
